(19) United States
(12) Patent Application Publication
(10) Pub. No.: US 2024/0366610 A1
(43) Pub. Date: Nov. 7, 2024

(54) METHODS AND COMPOSITIONS FOR TREATING CANCER

(71) Applicant: Onconova Therapeutics, Inc., Newtown, PA (US)

(72) Inventors: Steven Fruchtman, Newtown, PA (US); Mark S. Gelder, Newtown, PA (US); Matthew Parris, Newtown, PA (US)

(21) Appl. No.: 18/609,409

(22) Filed: Mar. 19, 2024

Related U.S. Application Data
(63) Continuation of application No. PCT/US22/45460, filed on Sep. 30, 2022.
(60) Provisional application No. 63/250,381, filed on Sep. 30, 2021.

(51) Int. Cl. A61K 31/519; A61K 31/198; A61P 35/00

(52) U.S. Cl. CPC A61K 31/519; A61K 31/198; A61P 35/00

(57) ABSTRACT

Disclosed herein is a method of treating a cancer in a subject in need thereof, the method comprising administering to the subject a therapeutically-effective amount of a first compound, or a pharmaceutically-acceptable salt or zwitterion thereof, and a therapeutically-effective amount of a second compound, or a pharmaceutically-acceptable salt thereof.

### CROSS-REFERENCE

The present application is a continuation of International Application No. PCT/US22/45460, filed Sep. 30, 2022, which claims the benefit of U.S. Provisional Application No. 63/250,381, filed Sep. 30, 2021, which is incorporated by reference herein in its entirety.

### BACKGROUND

Some cancers, for example, lung adenocarcinomas, have a KRAS mutation as a predominant genetic driver. Tumors carrying a mutation in KRAS can have a worse prognosis than KRAS wild-type tumors have. Therapies that can target the KRAS pathway could be beneficial for patients with cancers having KRAS mutations.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

Disclosed herein are methods of treating a cancer in a subject in need thereof.

In one aspect, disclosed herein is a method of treating a cancer in a subject in need thereof, the method comprising:
administering to the subject a therapeutically-effective amount of a first compound of formula (I)

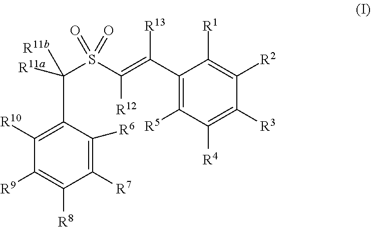

or a pharmaceutically-acceptable salt or zwitterion thereof,
   wherein:
each R¹, R², R³, R⁴, R⁵, R⁶, R⁷, R⁸, R⁹, R¹⁰, R^{11a}, R^{11b}, R¹², and R¹³ is independently alkyl, alkenyl, alkynyl, alkoxy, aryl, heteroaryl, heterocyclyl, C(O)R^{x}, C(O)OR^{x}, C(O)NR^{x}R^{y}, OR^{x}, SR^{x}, NR^{x}R^{y}, NR^{x}C(O)R^{y}, OC(O)R^{x}, or SiR^{x}R^{y}R^{z}, each of which is independently substituted or unsubstituted; or hydrogen or halogen; and
each R^{x}, R^{y}, and R^{z} is independently alkyl, alkenyl, alkynyl, aryl, heteroaryl, or heterocyclyl, each of which is independently substituted or unsubstituted; or hydrogen or halogen; and
administering to the subject a therapeutically-effective amount of a second compound of formula (II)

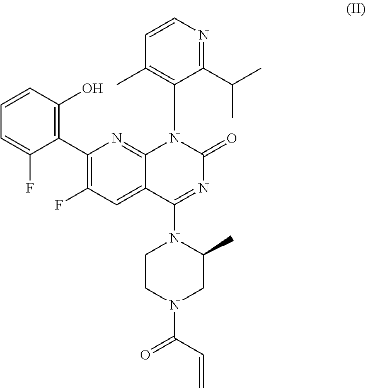 [SMILES: C=CC(=O)N1CCN(c2nc(=O)n(-c3c(C)ccnc3C(C)C)c3nc(-c4c(O)cccc4F)c(F)cc23)[C@@H](C)C1]

or a pharmaceutically-acceptable salt thereof,
wherein the cancer comprises a KRAS mutation.

In some embodiments, the KRAS mutation is KRASG12C, KRASG12D, or KRASG12V. In some embodiments, the cancer is a solid cancer. In some embodiments, the solid cancer is lung cancer, colorectal cancer, pancreatic cancer, or skin cancer.

In some embodiments, the cancer is non-small cell lung cancer. In some embodiments, the non-small cell lung cancer is KRAS positive non-small cell lung cancer. In some embodiments, the non-small cell lung cancer is locally advanced. In some embodiments, the non-small cell lung cancer is metastatic KRAS positive non-small cell lung cancer.

In some embodiments, the cancer is a relapsing or refractory cancer.

In some embodiments, the subject received a systemic therapy for the non-small cell lung cancer prior to the administering. In some embodiments, the subject received a systemic therapy for the non-small cell lung cancer followed by the second compound prior to the administering.

In some embodiments, the first compound and the second compound are administered simultaneously. In some embodiments, the first compound and the second compound are administered sequentially. In some embodiments, the first compound is administered prior to the second compound. In some embodiments, the second compound is administered prior to the first compound. In some embodiments, the administering the first compound and the administering the second compound are at least 24 hours apart.

In some embodiments, the therapeutically-effective amount of the first compound is about 100 mg per day to about 1400 mg per day. In some embodiments, the therapeutically-effective amount of the first compound is about 560 mg per day. In some embodiments, the therapeutically-effective amount of the first compound is about 840 mg per day. In some embodiments, the therapeutically-effective amount of the first compound is about 1,120 mg per day.

In some embodiments, the administering of the first compound is oral.

In some embodiments, the administering of the first compound is intravenous. In some embodiments, the administering of the first compound is a 72 hour intravenous infusion.

In some embodiments, the administering of the first compound is on day 1 to 3 of a two-week cycle.

In some embodiments, the administering of the first compound is on day 1 to 3 of a two-week cycle for 8 cycles, then on days 1 to 3 of a four-week cycle.

In some embodiments, the administering of the first compound is on a four-week cycle of daily for three weeks followed by one week with no administration.

In some embodiments, the administering of the first compound is oral on a four-week cycle of: i) three weeks of about 280 mg per morning and about 280 mg per evening; and ii) one week of no administration.

In some embodiments, the administering of the first compound is oral on a four-week cycle of: i) three weeks of about 560 mg per morning and about 280 mg per evening; and ii) one week of no administration. In some embodiments, the administering of the first compound is oral on a four-week cycle of: i) three weeks of about 560 mg per morning and about 560 mg per evening; and ii) one week of no administration. In some embodiments, the administering of the first compound is oral on a four-week cycle of: i) three weeks of about 840 mg per morning and about 560 mg per evening; and ii) one week of no administration. In some embodiments, the administering of the first compound is oral on a four-week cycle of: i) three weeks of about 840 mg per morning and about 840 mg per evening; and ii) one week of no administration.

In some embodiments, the administering occurs in a morning and an evening of a day, wherein the subject is in a fasted state for administering in the morning and in a fed state for administering in the evening.

In some embodiments, the first compound is administered on a morning and an afternoon of a day, wherein the therapeutically-effective amount of the first compound in the morning is about 150 mg/kg to about 300 mg/kg, and the therapeutically-effective amount of the first compound in the afternoon is about 100 mg/kg to about 250 mg/kg.

In some embodiments, the therapeutically-effective amount of the first compound is about 1,800 mg, and the about 1,800 mg is diluted in 0.9% saline.

In some embodiments, the administering of the second compound is oral. In some embodiments, the therapeutically-effective amount of the second compound is about 960 mg per day. In some embodiments, the administering of the second compound is once daily.

In some embodiments, the first compound and the second compound are each administered orally.

In some embodiments, the first compound is administered intravenously and the second compound is administered orally.

In some embodiments, the first compound is

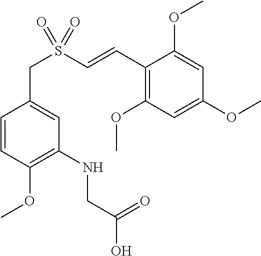

or a pharmaceutically-acceptable salt or zwitterion thereof.

In some embodiments, the first compound is

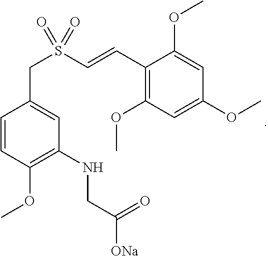

In another aspect, disclosed herein is a method of treating a non-small cell lung cancer in a subject in need thereof, the method comprising administering to the subject: a therapeutically-effective amount of a first compound, wherein the first compound is rigosertib or a pharmaceutically-acceptable salt or zwitterion thereof; and a therapeutically-effective amount of a second compound, wherein the second compound is sotorasib or a pharmaceutically-acceptable salt thereof.

### DETAILED DESCRIPTION

The present disclosure provides a method of treating a cancer in a subject in need thereof, the method comprising administering to the subject a therapeutically-effective amount of a first compound of formula (I):

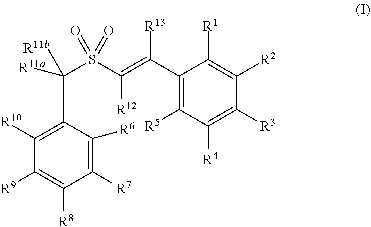

or a pharmaceutically-acceptable salt or zwitterion thereof,
wherein:
each R¹, R², R³, R⁴, R⁵, R⁶, R⁷, R⁸, R⁹, R¹⁰, R^{11a}, R^{11b}, R¹², and R¹³ is independently alkyl, alkenyl, alkynyl, alkoxy, aryl, heteroaryl, heterocyclyl, C(O)R^{x}, C(O)OR^{x}, C(O)NR^{x}R^{y}, OR^{x}, SR^{x}, NR^{x}R^{y}, NR^{x}C(O)R^{y}, OC(O)R^{x}, or SiR^{x}R^{y}R^{z}, each of which is independently substituted or unsubstituted; or hydrogen or halogen; and
each R^{x}, R^{y}, and R^{z} is independently alkyl, alkenyl, alkynyl, aryl, heteroaryl, or heterocyclyl, each of which is independently substituted or unsubstituted; or hydrogen or halogen, and
a therapeutically-effective amount of a second compound of formula II

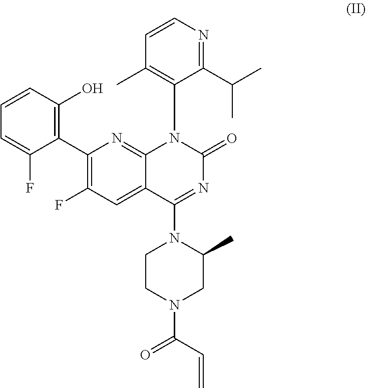 [SMILES: C=CC(=O)N1CCN(c2nc(=O)n(-c3c(C)ccnc3C(C)C)c3nc(-c4c(O)cccc4F)c(F)cc23)[C@@H](C)C1]

or a pharmaceutically-acceptable salt thereof,
wherein the cancer comprises a KRAS mutation.

The present disclosure also provides a method of treating a cancer, comprising administering to the subject a therapeutically-effective amount of a first compound of formula I, for example, (E)-2-(5-((2,4,6-trimethoxystyrylsulfonyl)methyl)-2-methoxyphenylamino)acetic acid, or a pharmaceutically-acceptable salt or zwitterion thereof, and a therapeutically-effective amount of a second compound, for example, 6-fluoro-7-(2-fluoro-6-hydroxyphenyl)-1-(4-methyl-2-propan-2-ylpyridin-3-yl)-4-[(2S)-2-methyl-4-prop-2-enoylpiperazin-1-yl]pyrido[2,3-d]pyrimidin-2-one, or a pharmaceutically-acceptable salt thereof.

Rigosertib ((E)-2-(5-((2,4,6-trimethoxystyrylsulfonyl)methyl)-2-methoxyphenylamino)acetic acid, or a pharmaceutically-acceptable salt or zwitterion thereof) can block the RAS cascade and promote the expression of novel antigens on the tumor's surface. Doing so can turn cold tumors that are not surrounded by host lymphocytes into hot tumors that are surrounded by host lymphocytes. Rigosertib can reduce AKT phosphorylation in squamous cell carcinoma keratinocytes without affecting normal primary keratinocytes.

Sotorasib (6-fluoro-7-(2-fluoro-6-hydroxyphenyl)-1-(4-methyl-2-propan-2-ylpyridin-3-yl)-4-[(2S)-2-methyl-4-prop-2-enoylpiperazin-1-yl]pyrido[2,3-d]pyrimidin-2-one, or a pharmaceutically-acceptable salt thereof) is an inhibitor of KRAS^{G12C}, a tumor-restricted, mutant-oncogenic form of the RAS GTPase, KRAS. Sotorasib is indicated for the treatment of adult patients with KRAS G12C-mutated, locally advanced or metastatic non-small cell lung cancer (NSCLC) in patients who received at least one prior systemic therapy.

### Compounds of the Disclosure

A compound disclosed herein can be of the formula (I):

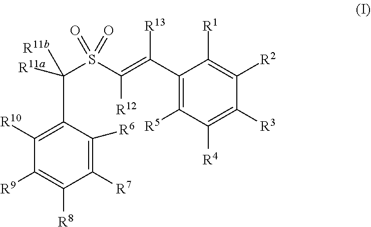

or a pharmaceutically-acceptable salt or zwitterion thereof,
   wherein:
each R¹, R², R³, R⁴, R⁵, R⁶, R⁷, R⁸, R⁹, R¹⁰, R^{11a}, R^{11b}, R¹², and R¹³ is independently alkyl, alkenyl, alkynyl, alkoxy, aryl, heteroaryl, heterocyclyl, C(O)R^{x}, C(O)OR^{x}, C(O)NR^{x}R^{y}, OR^{x}, SR^{x}, NR^{x}R^{y}, NR^{x}C(O)R^{y}, OC(O)R^{x}, or SiR^{x}R^{y}R^{z}, each of which is independently substituted or unsubstituted; or hydrogen or halogen; and
each R^{x}, R^{y}, and R^{z} is independently alkyl, alkenyl, alkynyl, aryl, heteroaryl, or heterocyclyl, each of which is independently substituted or unsubstituted; or hydrogen or halogen.

In some embodiments, the compound can be of the formula (Ia):

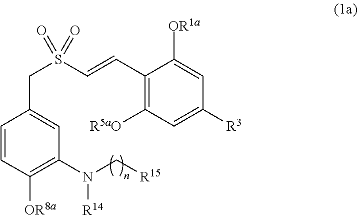

or a pharmaceutically-acceptable salt or zwitterion thereof
   wherein
R¹⁵ is C₁-C₆ alkyl, aryl, cycloalkyl, heteroaryl, heterocyclyl, —C(O)NR¹⁶R¹⁷, —C(NH)NR¹⁸R¹⁹, —CO₂R²⁰, —P(O)(OR²¹)₂, or —OP(O)(OR²²)₂;
R¹⁴ is hydrogen or C₁-C₆ alkyl;
each of R^{1a}, R^{5a} and R^{8a} is independently C₁-C₆ alkyl;
each of R¹⁶, R¹⁷, R¹⁸, R¹⁹, R²⁰, R²¹ and R²² is independently hydrogen or C₁-C₆ alkyl;
R³ is hydrogen, halogen, C₁-C₆ alkyl, C₁-C₆ alkoxy, or NR²³R²⁴;
each of R²³ and R²⁴ is hydrogen or C₁-C₆ alkyl; and
n is 1, 2, 3, 4, 5, or 6.

In some embodiments, R¹⁵ is —CO₂R²⁰. In some embodiments, R²⁰ is hydrogen. In some embodiments, R²⁰ is C₁-C₆ alkyl. In some embodiments, R¹⁴ is hydrogen. In some embodiments, each of R^{1a}, R^{5a} and R^{8a} is methyl. In some embodiments, R³ is C₁-C₆ alkoxy. In some embodiments, R³ is —OCH₃. In some embodiments, n is 1.

Compounds disclosed herein can include all stereoisomers, enantiomers, diastereomers, mixtures, racemates, atropisomers, and tautomers thereof.

Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, ureido groups, epoxy groups, and ester groups.

Non-limiting examples of alkyl and alkylene groups include straight, branched, and cyclic alkyl and alkylene groups. An alkyl or alkylene group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of substituted alkyl groups includes hydroxymethyl, chloromethyl, trifluoromethyl, aminomethyl, 1-chloroethyl, 2-hydroxyethyl, 1,2-difluoroethyl, and 3-carboxypropyl.

Non-limiting examples of cyclic alkyl groups include cyclopropyl, cyclobutyl, cyclopentyl, cyclohexyl, cycloheptlyl, and cyclooctyl groups. Cyclic alkyl groups also include fused-, bridged-, and spiro-bicycles and higher fused-, bridged-, and spiro-systems. A cyclic alkyl group can be substituted with any number of straight, branched, or cyclic alkyl groups. Non-limiting examples of cyclic alkyl groups include cyclopropyl, 2-methyl-cycloprop-1-yl, cycloprop-2-en-1-yl, cyclobutyl, 2,3-dihydroxycyclobut-1-yl, cyclobut-2-en-1-yl, cyclopentyl, cyclopent-2-en-1-yl, cyclopenta-2,4-dien-1-yl, cyclohexyl, cyclohex-2-en-1-yl, cycloheptyl, cyclooctanyl, 2,5-dimethylcyclopent-1-yl, 3,5-dichlorocyclohex-1-yl, 4-hydroxycyclohex-1-yl, 3,3,5-trimethylcyclohex-1-yl, octahydropentalenyl, octahydro-1H-indenyl, 3a,4,5,6,7,7a-hexahydro-3H-inden-4-yl, decahydroazulenyl, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

Non-limiting examples of alkenyl and alkenylene groups include straight, branched, and cyclic alkenyl groups. The olefin or olefins of an alkenyl group can be, for example, E, Z, cis, trans, terminal, or exo-methylene. An alkenyl or alkenylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkenyl and alkenylene groups include ethenyl, prop-1-en-1-yl, isopropenyl, but-1-en-4-yl; 2-chloroethenyl, 4-hydroxybuten-1-yl, 7-hydroxy-7-methyloct-4-en-2-yl, and 7-hydroxy-7-methyloct-3,5-dien-2-yl.

Non-limiting examples of alkynyl or alkynylene groups include straight, branched, and cyclic alkynyl groups. The triple bond of an alkylnyl or alkynylene group can be internal or terminal. An alkylnyl or alkynylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkynyl or alkynylene groups include ethynyl, prop-2-yn-1-yl, prop-1-yn-1-yl, and 2-methyl-hex-4-yn-1-yl; 5-hydroxy-5-methylhex-3-yn-1-yl, 6-hydroxy-6-methylhept-3-yn-2-yl, and 5-hydroxy-5-ethylhept-3-yn-1-yl.

A halo-alkyl group can be any alkyl group substituted with any number of halogen atoms, for example, fluorine, chlorine, bromine, and iodine atoms. A halo-alkenyl group can be any alkenyl group substituted with any number of halogen atoms. A halo-alkynyl group can be any alkynyl group substituted with any number of halogen atoms.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy.

An aryl group can be heterocyclic or non-heterocyclic. An aryl group can be monocyclic or polycyclic. An aryl group can be substituted with any number of substituents described herein, for example, hydrocarbyl groups, alkyl groups, alkoxy groups, and halogen atoms. Non-limiting examples of aryl groups include phenyl, toluyl, naphthyl, pyrrolyl, pyridyl, imidazolyl, thiophenyl, and furyl. Non-limiting examples of substituted aryl groups include 3,4-dimethylphenyl, 4-tert-butylphenyl, 4-cyclopropylphenyl, 4-diethylaminophenyl, 4-(trifluoromethyl)phenyl, 4-(difluoromethoxy)-phenyl, 4-(trifluoromethoxy)phenyl, 3-chlorophenyl, 4-chlorophenyl, 3,4-dichlorophenyl, 2-fluorophenyl, 2-chlorophenyl, 2-iodophenyl, 3-iodophenyl, 4-iodophenyl, 2-methylphenyl, 3-fluorophenyl, 3-methylphenyl, 3-methoxyphenyl, 4-fluorophenyl, 4-methylphenyl, 4-methoxyphenyl, 2,3-difluorophenyl, 3,4-difluorophenyl, 3,5-difluorophenyl, 2,3-dichlorophenyl, 3,4-dichlorophenyl, 3,5-dichlorophenyl, 2-hydroxyphenyl, 3-hydroxyphenyl, 4-hydroxyphenyl, 2-methoxyphenyl, 3-methoxyphenyl, 4-methoxyphenyl, 2,3-dimethoxyphenyl, 3,4-dimethoxyphenyl, 3,5-dimethoxyphenyl, 2,4-difluorophenyl, 2,5-difluorophenyl, 2,6-difluorophenyl, 2,3,4-trifluorophenyl, 2,3,5-trifluorophenyl, 2,3,6-trifluorophenyl, 2,4,5-trifluorophenyl, 2,4,6-trifluorophenyl, 2,4-dichlorophenyl, 2,5-dichlorophenyl, 2,6-dichlorophenyl, 3,4-dichlorophenyl, 2,3,4-trichlorophenyl, 2,3,5-trichlorophenyl, 2,3,6-trichlorophenyl, 2,4,5-trichlorophenyl, 3,4,5-trichlorophenyl, 2,4,6-trichlorophenyl, 2,3-dimethylphenyl, 2,4-dimethylphenyl, 2,5-dimethylphenyl, 2,6-dimethylphenyl, 2,3,4-trimethylphenyl, 2,3,5-trimethylphenyl, 2,3,6-trimethylphenyl, 2,4,5-trimethylphenyl, 2,4,6-trimethylphenyl, 2-ethylphenyl, 3-ethylphenyl, 4-ethylphenyl, 2,3-diethylphenyl, 2,4-diethylphenyl, 2,5-diethylphenyl, 2,6-diethylphenyl, 3,4-diethylphenyl, 2,3,4-triethylphenyl, 2,3,5-triethylphenyl, 2,3,6-triethylphenyl, 2,4,5-triethylphenyl, 2,4,6-triethylphenyl, 2-isopropylphenyl, 3-isopropylphenyl, and 4-isopropylphenyl.

Non-limiting examples of substituted aryl groups include 2-aminophenyl, 2-(N-methylamino)phenyl, 2-(N,N-dimethylamino)phenyl, 2-(N-ethylamino)phenyl, 2-(N,N-diethylamino)phenyl, 3-aminophenyl, 3-(N-methylamino)phenyl, 3-(N,N-dimethylamino)phenyl, 3-(N-ethylamino)phenyl, 3-(N,N-diethylamino)phenyl, 4-aminophenyl, 4-(N-methylamino)phenyl, 4-(N,N-dimethylamino)phenyl, 4-(N-ethylamino)phenyl, and 4-(N,N-diethylamino)phenyl.

A heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic. Non-limiting examples of heterocycles include pyrrole, pyrrolidine, pyridine, piperidine, succinamide, maleimide, morpholine, imidazole, thiophene, furan, tetrahydrofuran, pyran, and tetrahydropyran.

Non-limiting examples of heterocycles include: heterocyclic units having a single ring containing one or more heteroatoms, non-limiting examples of which include, diazirinyl, aziridinyl, azetidinyl, pyrazolidinyl, imidazolidinyl, oxazolidinyl, isoxazolinyl, thiazolidinyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl, 2,3,4,5-tetrahydro-1H-azepinyl, 2,3-dihydro-1H-indole, and 1,2,3,4-tetrahydroquinoline; and ii) heterocyclic units having 2 or more rings one of which is a heterocyclic ring, non-limiting examples of which include hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, and decahydro-1H-cycloocta[b]pyrrolyl.

Non-limiting examples of heteroaryl include: i) heteroaryl rings containing a single ring, non-limiting examples of which include, 1,2,3,4-tetrazolyl, [1,2,3]triazolyl, [1,2,4]triazolyl, triazinyl, thiazolyl, 1H-imidazolyl, oxazolyl, isoxazolyl, isothiazolyl, furanyl, thiophenyl, pyrimidinyl, 2-phenylpyrimidinyl, pyridinyl, 3-methylpyridinyl, and 4-dimethylaminopyridinyl; and ii) heteroaryl rings containing 2 or more fused rings one of which is a heteroaryl ring, non-limiting examples of which include: 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 4,5,6,7-tetrahydro-1-H-indolyl, quinoxalinyl, quinazolinyl, quinolinyl, 8-hydroxy-quinolinyl, and isoquinolinyl.

A compound disclosed herein can be of the formula (Ib):

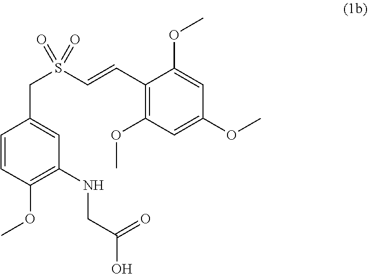 [SMILES: COc1cc(OC)c(/C=C/S(=O)(=O)Cc2ccc(OC)c(NCC(=O)O)c2)c(OC)c1]

(E)-2-(5-((2,4,6-trimethoxystyrylsulfonyl)methyl)-2-methoxyphenylamino)acetic acid, or a pharmaceutically-acceptable salt or zwitterion thereof.

In some embodiments, a compound disclosed herein is a sodium salt.

A compound disclosed herein can be of the formula (Ic):

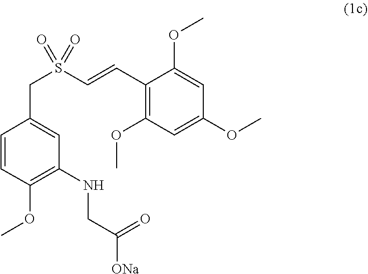

sodium (E)-2-(5-((2,4,6-trimethoxystyrylsulfonyl)methyl)-2-methoxyphenylamino)acetate.

(E)-2-(5-((2,4,6-trimethoxystyrylsulfonyl)methyl)-2-methoxyphenylamino)acetic acid, or a pharmaceutically-acceptable salt or zwitterion thereof can be referred to as Rigosertib.

A compound disclosed herein can be of the formula (II):

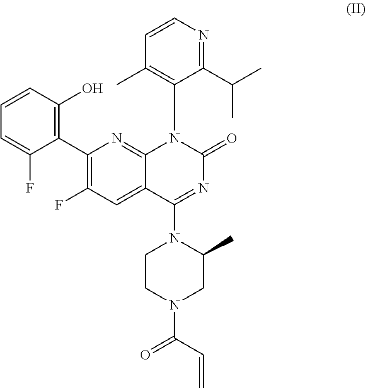 [SMILES: C=CC(=O)N1CCN(c2nc(=O)n(-c3c(C)ccnc3C(C)C)c3nc(-c4c(O)cccc4F)c(F)cc23)[C@@H](C)C1]

6-fluoro-7-(2-fluoro-6-hydroxyphenyl)-1-(4-methyl-2-propan-2-ylpyridin-3-yl)-4-[(2S)-2-methyl-4-prop-2-enoylpiperazin-1-yl]pyrido[2,3-d]pyrimidin-2-one, or a pharmaceutically-acceptable salt thereof.

6-fluoro-7-(2-fluoro-6-hydroxyphenyl)-1-(4-methyl-2-propan-2-ylpyridin-3-yl)-4-[(2S)-2-methyl-4-prop-2-enoylpiperazin-1-yl]pyrido[2,3-d]pyrimidin-2-one, or a pharmaceutically-acceptable salt thereof can be referred to as Sotorasib.

### Pharmaceutically Acceptable Salts

The method disclosed herein provides the use of pharmaceutically-acceptable salts of any compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is a sodium salt.

Metal salts can arise from the addition of an inorganic base to a compound disclosed herein. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Acid addition salts can arise from the addition of an acid to a compound disclosed herein. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate salt, an ethanesulfonate salt, a benzenesulfonate salt, a p-toluenesulfonate salt, a citrate salt, an oxalate salt, or a maleate salt.

### Methods of Treatment

A mechanism of action for a compound disclosed herein, for example, rigosertib, is to down-modulate the mutated RAS pathway. This modulation of the RAS pathway can result in less production of ERK, a protein involved in cell proliferation. When ERK is overexpressed, ERK can provide a proliferative advantage to cells, thus leading to cancer.

Rigosertib ((E)-2-(5-((2,4,6-trimethoxystyrylsulfonyl)methyl)-2-methoxyphenylamino)acetic acid), or a pharmaceutically-acceptable salt or zwitterion thereof) can block the RAS cascade and promote the expression of novel antigens on the tumor's surface. Doing so can turn cold tumors that are not surrounded by host lymphocytes into hot tumors that are surrounded by host lymphocytes.

Sotorasib 6-fluoro-7-(2-fluoro-6-hydroxyphenyl)-1-(4-methyl-2-propan-2-ylpyridin-3-yl)-4-[(2S)-2-methyl-4-prop-2-enoylpiperazin-1-yl]pyrido[2,3-d]pyrimidin-2-one), or a pharmaceutically-acceptable salt or zwitterion thereof), is an inhibitor of KRAS^{G12C}, a tumor-restricted, mutant-oncogenic form of the RAS GTPase, KRAS. Sotorasib irreversibly forms a covalent bond with the unique cysteine residue of KRAS^{G12C}, locking the protein in an inactive state that prevents downstream signaling without affecting wild-type KRAS. Sotorasib blocks KRAS signaling, inhibits cell growth, and promotes apoptosis only in KRAS G12C tumor cell lines. Sotorasib inhibits KRAS^{G12C} in vitro and in vivo with minimal detectable off-target activity. In mouse tumor xenograft models, sotorasib treatment leads to tumor regressions and prolonged survival and is associated with anti-tumor immunity in KRAS G12C models.

Provided herein is method of treating non-small cell lung cancer in a subject in need thereof, the method comprising administering to the subject: a therapeutically-effective amount of a first compound, wherein the first compound is rigosertib or a pharmaceutically-acceptable salt or zwitterion thereof; and a therapeutically-effective amount of a second compound, wherein the second compound is sotorasib or a pharmaceutically-acceptable salt thereof.

Provided herein is a method of treating a cancer in a subject in need thereof, wherein the cancer comprises a KRAS mutation. In some embodiments, the KRAS mutation is KRASG12C, KRASG12D, or KRASG12V. In some embodiments, the cancer is a solid cancer. Non-limiting examples of solid cancers comprise lung cancer, colorectal cancer, pancreatic cancer, and skin cancer.

In some embodiments, the cancer is non-small cell lung cancer. In some embodiments, the non-small cell lung cancer is KRAS positive non-small cell lung cancer. In some embodiments, the non-small cell lung cancer is locally advanced. In some embodiments, the cancer is a relapsing or refractory cancer.

Provided herein is a method of treating advanced KRAS+ non-small cell lung cancer NSCLC with G12C mutations in a subject in need thereof, the method comprising administering to the subject: a therapeutically-effective amount of a first compound, wherein the first compound is rigosertib or a pharmaceutically-acceptable salt or zwitterion thereof, and a therapeutically-effective amount of a second compound, wherein the second compound is sotorasib or a pharmaceutically-acceptable salt thereof.

In some embodiments, the subject has KRAS G12C-mutated locally advanced or metastatic NSCLC, as determined by a FDA-approved test, who have received and failed at least one prior systemic therapy followed by a failure on Sotorasib. In some embodiments, the subject has KRAS G12C-mutated locally advanced or metastatic NSCLC, as determined by a FDA-approved test, who have received and failed at least one prior systemic therapy (Sotorasib naïve). In some embodiments, combination of sotorasib with rigosertib provides a response rate greater than that obtained with sotorasib monotherapy.

FDA-approved tests for detection of KRAS G12C mutations are available and include, for example, therascreen EGFR RGQ PCR Kit, cobas EGFR Mutation Test v2, PD-L1 TIC 22C3 pharmDx, FoundationOne CDx, Ventana ALK (D5F3) CDx Assay, Oncomine Dx Target Test, therascreen KRAS RGQ PCR Kit, Vysis ALK Break Apart FISH Probe Kit, PD-L1 IHC 28-8 pharmDx, Guardant 360® CDx, FoundationOne® Liquid CDx, ONCO/Reveal Dx Lunch, and Colon Cancer Assay (O/RDx-LCCA).

### Pharmaceutical Compositions of the Disclosure

### Rigosertib Intravenous (IV) Formulation

A pharmaceutical formulation disclosed herein can be, for example, a parenteral formulation of a solution of the active pharmaceutical ingredient (API) at a concentration of about 75 mg/mL in a liquid vehicle of polyethylene glycol (PEG) 400 with pH 7-13 or pH 12-13 adjusted by use of sodium hydroxide (NaOH) solution. The solution can be a clear, colorless to pale yellow, sterile, preservative-free solution packaged in a clear glass vial sealed with a Teflon-coated rubber stopper. A vial disclosed herein can be a 30-mL clear glass vial containing, for example, 24 mL of the parenteral formulation. Dilution with IV saline can be required when the parenteral formulation is administered IV.

### Rigosertib Oral Capsule Formulation

A compound disclosed herein, for example, rigosertib, can be formulated as a soft gelatin capsules in two strengths: 1) about 70 mg/mL solution of rigosertib in PEG 400; or 2) about 280 mg/mL suspension of rigosertib in PEG 400 and PEG 4000. The 70 mg capsules contain 1 mL of a 70 mg/mL solution of rigosertib and can be, for example, clear, transparent, and oblong. The 280 mg capsules contain 1 mL of a 280 mg/mL solution of rigosertib and can be, for example, of an opaque yellow-orange color.

### Sotorasib Oral Tablet Formulation

A compound disclosed herein, for example, sotorasib, can be formulated as tablets for oral use. Sotorasib tablets can be film coated. In some embodiments, sotorasib can be formulated as film-coated tablets containing 120 mg of sotorasib. Inactive ingredients in the tablet core can be microcrystalline cellulose, lactose monohydrate, croscarmellose sodium, and magnesium stearate. The film coating material can comprise polyvinyl alcohol, titanium dioxide, polyethylene glycol, talc, and iron oxide yellow. The 120 mg tablets can be, for example, yellow, oblong-shaped, film-coated tablets.

### Pharmaceutical Excipients

A pharmaceutical composition of a compound disclosed herein can be a combination of any pharmaceutical compounds described herein with other chemical components, such as carriers, stabilizers, diluents, dispersing agents, suspending agents, thickening agents, or excipients. The pharmaceutical composition facilitates administration of the compound to an organism. Pharmaceutical compositions can be administered in therapeutically-effective amounts as pharmaceutical compositions by various forms and routes including, for example, intravenous, intravitreal, intranasal, inhalation, nasal inhalation, mouth inhalation, intratracheal, intrapulmonary, transmucosal, subcutaneous, intramuscular, oral, rectal, aerosol, parenteral, ophthalmic, pulmonary, transdermal, vaginal, otic, nasal, and topical administration.

A pharmaceutical composition can be administered in a local or systemic manner, for example, via injection of the compound directly into an organ, optionally in a depot or sustained release formulation. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated readily by combining the active compounds with pharmaceutically-acceptable carriers or excipients. Such carriers can be used to formulate tablets, powders, pills, dragees, capsules, liquids, gels, syrups, elixirs, slurries, or suspensions for oral ingestion by a subject.

Pharmaceutical preparations for oral use can be obtained by mixing one or more solid excipients with one or more compounds described herein, optionally grinding the resulting mixture, and processing the mixture of granules, after adding suitable auxiliaries, if desired, to obtain tablets or dragee cores. Cores can be provided with suitable coatings. For this purpose, concentrated sugar solutions can be used. The sugar solutions can contain an excipient such as polyvinyl alcohol, gum arabic, talc, polyvinylpyrrolidone, carbopol gel, polyethylene glycol, titanium dioxide, lacquer solutions, iron oxide, and suitable organic solvents or solvent mixtures. Dyestuffs or pigments can be added to the tablets or dragee coatings, for example, for identification or to characterize different combinations of active compound doses.

Pharmaceutical preparations that can be used orally include push-fit capsules made of gelatin, and sealed capsules made of gelatin and a plasticizer, such as glycerol or sorbitol. In some embodiments, the capsule comprises a hard gelatin capsule comprising one or more of pharmaceutical, bovine, and plant gelatins. A gelatin can be alkaline-processed. The push-fit capsules can contain the active ingredients in admixture with filler such as lactose, binders such as starches, or lubricants such as talc or magnesium stearate, and stabilizers. In soft capsules, the active compounds can be dissolved or suspended in suitable liquids, such as fatty oils, liquid paraffin, or liquid polyethylene glycols. Stabilizers can be added. All formulations for oral administration are provided in dosages suitable for such administration.

For buccal or sublingual administration, the compositions can be tablets, lozenges, or gels.

Parenteral injections can be formulated for bolus injection or continuous infusion. The pharmaceutical compositions can be in a form suitable for parenteral injection such as a sterile suspension, solution, or emulsion in oily or aqueous vehicles, and can contain formulatory agents such as suspending, stabilizing, or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, synthetic fatty acid esters, such as ethyl oleate or triglycerides, and liposomes. Aqueous injection suspensions can contain substances that increase the viscosity of the suspension, such as sodium carboxymethyl cellulose, sorbitol, and dextran. The suspension can also contain suitable stabilizers or agents that increase the solubility of the compounds to allow for the preparation of highly concentrated solutions. Alternatively, the active ingredient can be in powder form for constitution with a suitable vehicle, e.g., sterile pyrogen-free water, before use.

An active compound can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers, and preservatives.

Formulations suitable for transdermal administration of the active compounds can employ transdermal delivery devices and transdermal delivery patches, and can be lipophilic emulsions or buffered aqueous solutions, dissolved or dispersed in a polymer or an adhesive. Such patches can be constructed for continuous, pulsatile, or on demand delivery of pharmaceutical compounds. Transdermal delivery can be accomplished by iontophoretic patches. Additionally, transdermal patches can provide controlled delivery. The rate of absorption can be slowed by using rate-controlling membranes or by trapping the compound within a polymer matrix or gel. Conversely, absorption enhancers can be used to increase absorption. An absorption enhancer or carrier can include absorbable, pharmaceutically-acceptable solvents to assist passage through the skin. For example, transdermal devices can be in the form of a bandage comprising a backing member, a reservoir containing compounds and carriers, a rate controlling barrier to deliver the compounds to the skin of the subject at a controlled and predetermined rate over a prolonged period of time, and adhesives to secure the device to the skin or the eye.

For administration by inhalation, the active compounds can be in a form as an aerosol, a vapor, a mist, or a powder. Inhalation can occur through by nasal delivery, oral delivery, or both.

Nasal or intranasal administration involves insufflation of compounds through the nose, for example, nasal drops and nasal sprays. This route of administration can result in local and/or systemic effects. Inhaler or insufflator devices can be used for nose-to-lung delivery of compounds described herein.

The compounds can also be formulated in rectal compositions such as enemas, rectal gels, rectal foams, rectal aerosols, suppositories, jelly suppositories, or retention enemas containing conventional suppository bases such as cocoa butter or other glycerides, and synthetic polymers such as polyvinylpyrrolidone and PEG. In suppository forms of the compositions, a low-melting point wax such as a mixture of fatty acid glycerides or cocoa butter, can be used.

In practicing a method of treatment or use provided herein, therapeutically-effective amounts of a compound described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a compound described herein can be manufactured, for example, by mixing, dissolving, granulating, dragee-making, levigating, emulsifying, encapsulating, entrapping, or compression processes.

The pharmaceutical compositions can include at least one pharmaceutically-acceptable carrier, diluent, or excipient and compound described herein as neutral or pharmaceutically-acceptable salt form. The methods and pharmaceutical compositions described herein can include the use of crystalline forms (also known as polymorphs), and active metabolites of these compounds having the same type of activity.

Methods for the preparation of compositions comprising a compound described herein include formulating a compound with one or more inert, pharmaceutically-acceptable excipients or carriers to form a solid, semi-solid, or liquid composition. Solid compositions include, for example, powders, tablets, dispersible granules, capsules, cachets, and suppositories. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions, and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in a method disclosed herein include feed, food, pellet, lozenge, liquid, elixir, aerosol, inhalant, spray, powder, tablet, pill, capsule, gel, geltab, nanosuspension, nanoparticle, microgel, suppository troches, aqueous or oily suspensions, ointment, patch, lotion, dentifrice, emulsion, creams, drops, dispersible powders or granules, emulsion in hard or soft gel capsules, syrups, phytoceuticals, nutraceuticals, and any combination thereof.

Non-limiting examples of pharmaceutically-acceptable excipients suitable for use in the method disclosed herein include fillers/diluents, granulating agents, binding agents, lubricating agents, disintegrating agents, moisture-barrier agents, flavoring agents, e.g., sweetening agents, glidants, anti-adherents, anti-static agents, surfactants, anti-oxidants, gums, coating agents, pigment/coloring agents, flavoring agents, coating agents, plasticizers, preservatives, suspending agents, emulsifying agents, anti-microbial agents, plant cellulosic material and spheronization agents, and any combination thereof.

Non-limiting examples of pharmaceutically-acceptable fillers/diluents include cellulose derivatives including microcrystalline cellulose, silicified microcrystalline cellulose carboxymethyl cellulose, methyl cellulose, hydroxypropyl cellulose, hydroxypropyl methylcellulose, ethyl cellulose, starches, sugars such as mannitol, sucrose, lactose, sorbitol, or dextrins (e.g., maltodextrin), and amino-sugars.

Non-limiting examples of pharmaceutically-acceptable binders include microcrystalline cellulose, gum tragacanth, gelatin, polyvinylpyrrolidone, copovidone, hydroxypropyl methylcellulose, and starch.

Non-limiting examples of pharmaceutically-acceptable disintegrants include croscarmellose sodium, sodium carboxymethyl starch, and crospovidone.

Non-limiting examples of pharmaceutically-acceptable lubricants include stearates such as magnesium stearate or zinc stearate, stearic acid, sodium stearyl fumarate, talc, glyceryl behenate, sodium lauryl sulfate, polyethylene glycol, and hydrogenated vegetable oil.

Non-limiting examples of pharmaceutically-acceptable glidants include colloidal silicon dioxide, talc, tribasic calcium phosphate, calcium silicate, cellulose, magnesium silicate, magnesium trisilicate, starch, magnesium stearate, talc, and mineral oil.

Non-limiting examples of moisture barrier agents include stearic acid.

Non-limiting examples of pharmaceutically-acceptable plasticizers include triethyl citrate.

Non-limiting examples of pharmaceutically-acceptable surfactants include sodium laurylsulfate or polysorbates, polyvinyl alcohol (PVA), polyethylene glycols, polyoxyethylene-polyoxypropylene block copolymers known as poloxamer, polyglycerin fatty acid esters such as decaglyceryl monolaurate and decaglyceryl monomyristate, sorbitan fatty acid ester such as sorbitan monostearate, polyoxyethylene sorbitan fatty acid ester such as polyoxyethylene sorbitan monooleate (Tween), polyethylene glycol fatty acid ester such as polyoxyethylene monostearate, polyoxyethylene alkyl ether such as polyoxyethylene lauryl ether, polyoxyethylene castor oil, and hardened castor oil such as polyoxyethylene hardened castor oil.

Non-limiting examples of pharmaceutically-acceptable flavoring agents include sweeteners such as sucralose and synthetic flavor oils and flavoring aromatics, natural oils, extracts from plants, leaves, flowers, fruits, and combinations thereof. Non-limiting examples of flavoring agents include cinnamon oils, oil of wintergreen, peppermint oils, clover oil, hay oil, anise oil, eucalyptus, peppermint, vanilla, citrus oil such as lemon oil, orange oil, grape and grapefruit oil, and fruit essences including apple, peach, pear, strawberry, raspberry, cherry, plum, pineapple, and apricot.

Non-limiting examples of pharmaceutically-acceptable pigments or colorants include alumina (dried aluminum hydroxide), annatto extract, calcium carbonate, canthaxanthin, caramel, β-carotene, cochineal extract, carmine, potassium sodium copper chlorophyllin (chlorophyllin-copper complex), dihydroxyacetone, bismuth oxychloride, synthetic iron oxide, ferric ammonium ferrocyanide, ferric ferrocyanide, chromium hydroxide green, chromium oxide greens, guanine, mica-based pearlescent pigments, pyrophyllite, mica, dentifrices, talc, titanium dioxide, aluminum powder, bronze powder, copper powder, and zinc oxide.

Non-limiting examples of buffering or pH adjusting agents include acidic buffering agents such as short chain fatty acids, citric acid, acetic acid, hydrochloric acid, sulfuric acid and fumaric acid; and basic buffering agents such as tris, sodium carbonate, sodium bicarbonate, sodium hydroxide, potassium hydroxide, and magnesium hydroxide.

Non-limiting examples of tonicity enhancing agents include ionic and non-ionic agents such as, alkali metal or alkaline earth metal halides, urea, glycerol, sorbitol, mannitol, propylene glycol, and dextrose.

Non-limiting examples of wetting agents include glycerin, cetyl alcohol, and glycerol monostearate.

Non-limiting examples of preservatives include benzalkonium chloride, benzoxonium chloride, thiomersal, phenylmercuric nitrate, phenylmercuric acetate, phenylmercuric borate, methylparaben, propylparaben, chlorobutanol, benzyl alcohol, phenyl alcohol, chlorohexidine, and polyhexamethylene biguanide.

Non-limiting examples of antioxidants include sorbic acid, ascorbic acid, ascorbate, glycine, α-tocopherol, butylated hydroxyanisole (BHA), and butylated hydroxytoluene (BHT).

A composition of a compound disclosed herein can be, for example, an immediate release form or a controlled release formulation. An immediate release formulation can be formulated to allow a compound to act rapidly. Non-limiting examples of immediate release formulations include readily dissolvable formulations. A controlled release formulation can be a pharmaceutical formulation that has been adapted such that drug release rates and drug release profiles can be matched to physiological and chronotherapeutic requirements or, alternatively, has been formulated to effect release of a drug at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

The disclosed compositions can optionally comprise from about 0.001% to about 0.005% weight by volume pharmaceutically-acceptable preservatives.

In some, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16, or about 24 hours.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

A method disclosed herein includes, for example, administration of a compound disclosed herein, or a pharmaceutically-acceptable salt thereof, in combination with a pharmaceutically-acceptable carrier. The carrier can be selected to minimize any degradation of the active ingredient and to minimize any adverse side effects in the subject.

A compound disclosed herein or a pharmaceutically-acceptable salt thereof disclosed herein can be conveniently formulated into pharmaceutical compositions composed of one or more pharmaceutically-acceptable carriers. See e.g., Remington's Pharmaceutical Sciences, latest edition, by E. W. Martin Mack Pub. Co., Easton, PA, which discloses carriers and methods of preparing pharmaceutical compositions that can be used in conjunction with the preparation of formulations of the compound described herein and which is incorporated by reference herein. Such pharmaceuticals can be standard carriers for administration of compositions to humans and non-humans, including solutions such as sterile water, saline, and buffered solutions at physiological pH. Other compositions can be administered according to standard procedures. For example, pharmaceutical compositions can also include one or more additional active ingredients such as antimicrobial agents, anti-inflammatory agents, and anesthetics.

Non-limiting examples of pharmaceutically-acceptable carriers include saline solution, Ringer's solution, and dextrose solution. The pH of the solution can be from about 5 to about 8, and can be from about 7 to about 7.5. Further carriers include sustained release preparations such as semipermeable matrices of solid hydrophobic polymers containing the compound disclosed herein or a pharmaceutically-acceptable salt thereof, wherein the matrices are in the form of shaped articles, such as films, liposomes, microparticles, and microcapsules.

A method disclosed herein relates to administering the compound disclosed herein or a pharmaceutically-acceptable salt or zwitterion thereof as part of a pharmaceutical composition. In some embodiments, compositions of a compound disclosed herein can comprise a liquid having an active agent in solution, in suspension, or both. Liquid compositions can include gels. In some embodiments, the liquid composition is aqueous. Alternatively, the composition can be an ointment. In some embodiments, the composition is an in situ gellable aqueous composition. In some embodiments, the composition is an in situ gellable aqueous solution.

Pharmaceutical formulations can include additional carriers, thickeners, diluents, buffers, preservatives, and surface active agents in addition to a compound disclosed herein. Pharmaceutical formulations can also include one or more additional active ingredients such as antimicrobial agents, anti-inflammatory agents, and anesthetics.

An excipient can fill a role as simple and direct as being an inert filler, or an excipient as used herein can be part of a pH stabilizing system or coating to insure delivery of the ingredients safely to the stomach.

The compound disclosed herein or a pharmaceutically-acceptable salt thereof can also be present in liquids, emulsions, or suspensions for delivery of active therapeutic agents in aerosol form to cavities of the body such as the nose, throat, or bronchial passages.

Depending on the intended mode of administration, the pharmaceutical compositions administered as part of a method disclosed herein can be in the form of solid, semi-solid, or liquid dosage forms, such as, for example, tablets, suppositories, pills, capsules, powders, liquids, suspensions, lotions, creams, gels, for example, in unit dosage form suitable for single administration of a precise dosage. The compositions can contain, as noted above, an effective amount of the compound disclosed herein or a pharmaceutically-acceptable salt thereof in combination with a pharmaceutically-acceptable carrier and, in addition, can include other medicinal agents, pharmaceutical agents, carriers, adjuvants, diluents, etc.

A pharmaceutically-acceptable excipient can be present in a pharmaceutical composition at a mass of between about 0.1% and about 99% by mass of the composition. For example, a pharmaceutically-acceptable excipient can be present in a pharmaceutical composition at a mass of between about 0.1% and about 95%, between about 0.1% and about 90%, between about 0.1% and about 85%, between about 0.1% and about 80%, between about 0.1% and about 75%, between about 0.1% and about 70%, between about 0.1% and about 65%, between about 0.1% and about 60%, between about 0.1% and about 55%, between about 0.1% and about 50%, between about 0.1% and about 45%, between about 0.1% and about 40%, between about 0.1% and about 35%, between about 0.1% and about 30%, between about 0.1% and about 25%, between about 0.1% and about 20%, between about 0.1% and about 15%, between about 0.1% and about 10%, between about 0.1% and about 5%, between about 0.1% and about 1%, by mass of the formulation.

A pharmaceutically-acceptable excipient can be present at about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% by mass of the formulation.

### Dosing

The first compound and the second compound can be administered simultaneously. The first compound and the second compound can be administered sequentially. For example, the first compound can be administered prior to the second compound. Alternatively, the second compound can be administered prior to the first compound. In some embodiments, a first administration and a second administration are at least 24 hours apart.

In some embodiments, the therapeutically-effective amount of the first compound is about 100, about 150, about 200, about 250, about 300, about 400, about 500, about 600, about 700, about 800, about 900, about 1000, about 1100, about 1200, about 1300, or about 1400 mg per day.

Oral rigosertib: A dosing regimen disclosed herein can be, for example, 280 mg of oral Rigosertib (e.g., 1 capsule of 280 mg) in the morning and 280 mg of oral Rigosertib (e.g., 1 capsule of 280 mg) in the afternoon, for a total of 560 mg/day. A dosing regimen disclosed herein can be, for example, 560 mg of oral Rigosertib (e.g., 2 capsules of 280 mg) in the morning and 280 mg of oral Rigosertib (e.g., 1 capsule of 280 mg) in the afternoon, for total of 840 mg/day. A dosing regimen disclosed herein can be, for example, 560 mg of oral Rigosertib (e.g., 2 capsules of 280 mg) in the morning and 560 mg of oral Rigosertib (e.g., 2 capsules of 280 mg) in the afternoon, for a total of 1120 mg/day. A dosing regimen disclosed herein can be, for example, one dose of 840 mg of oral rigosertib in the morning and 560 mg in the afternoon, for a total of 1,400 mg/day. A dosing regimen disclosed herein can be, for example, one dose of 840 mg of oral rigosertib in the morning and 840 mg in the afternoon, for a total of 1,680 mg/day.

In combination with a dose of Sotorasib (e.g., 960 mg per day), a 3+3 dose escalation can be incorporated to find the RP2D (recommended phase 2 dose) of Rigosertib. Dose escalation can continue by, for example, 280 mg increments. For example, a morning dose can be increased by 280 mg, and then the afternoon dose can be increased by 280 mg. Study subjects can take oral rigosertib continuously for a total of 3 weeks, every four week cycle (three weeks on, one week off drug).

Dose escalation can continue, depending on the observed number of dose limiting toxicities (DLT) in a single cohort. At that point, a prior dose cohort can be expanded to 6 patients and if less than 2 DLTs occur in those 6 patients, then that dose can be considered the MTD. Alternatively, the dose escalation can occur in 70 mg increments. Additionally, a much larger percentage of the total daily dose can be administered in the morning dose and a lower percentage in the afternoon dose (i.e. 1,400 mg in the a.m. and 560 mg in the afternoon).

### Intravenous Rigosertib:

A dosing regimen for intravenous rigosertib can be, for example, 1,800 mg/day, given on days 1-3 of a 14-day cycle for 8 cycles and then every on days 1-3 of a 28-day cycle thereafter. In combination with a dose of Sotorasib (e.g., 960 mg per day), a 3+3 dose escalation can be incorporated to find the RP2D (recommended phase 2 dose) of Rigosertib. Dose escalation cohorts can receive the full dose of Sotorasib and initially reduced doses of rigosertib, for example, 1,200 mg/day, then 1500 mg/day, then 1800 mg/day given on days 1-3 of a 14-day cycle for 8 cycles and then every on days 1-3 of a 28-day cycle thereafter. In solid tumor studies, where there is less risk of bone marrow toxicity, the dose can be further incrementally increased to 2,100 mg/day, 2,400 mg/day until the MTD and RP2D are established according to 3+3 dose escalation.

### Oral Sotorasib:

A dosing regimen disclosed herein can be, for example, once a day, twice a day, thrice a day, once a week, twice a week, or thrice a week. In some embodiments, a suitable amount Sotorasib can range from about 500 mg to about 1,000 mg per day, for example about 500 mg per day, about 550 mg per day, about 600 mg per day, about 650 mg per day, about 700 mg per day, about 750 mg per day, about 800 mg per day, about 850 mg per day, about 900 mg per day, about 910 mg per day, about 920 mg per day, about 930 mg per day, about 940 mg per day, about 950 mg per day, about 960 mg per day, about 970 mg per day, about 980 mg per day, about 990 mg per day, or about 1,000 mg per day. In some embodiments, a dosing regimen can be, for example, a dose of 960 mg orally once daily.

A compound disclosed herein can be administered via subcutaneous or intravenous injection. The volume of an injection can be about 0.1 mL, about 0.2 mL, about 0.3 mL, about 0.4 mL, about 0.5 mL, about 0.6 mL, about 0.7 mL, about 0.8 mL, about 0.9 mL, about 1 mL, about 1.1 mL, about 1.2 mL, about 1.3 mL, about 1.4 mL, about 1.5 mL, about 1.6 mL, about 1.7 mL, about 1.8 mL, about 1.9 mL, about 2 mL, about 2.1 mL, about 2.2 mL, about 2.3 mL, about 2.4 mL, about 2.5 mL, about 2.6 mL, about 2.7 mL, about 2.8 mL, about 2.9 mL, or about 3 mL. The individual dose administered to a subject can be about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for parenteral injection can be presented in unit dosage form, for example, in ampoules, or in multi-dose containers with a preservative.

A compound described herein can be present in a composition in a range of from about 1 mg to about 5 mg, from about 5 mg to about 10 mg, from about 10 mg to about 15 mg, from about 15 mg to about 20 mg, from about 20 mg to about 25 mg, from about 25 mg to about 30 mg, from about 30 mg to about 35 mg, from about 35 mg to about 40 mg, from about 40 mg to about 45 mg, from about 45 mg to about 50 mg, from about 50 mg to about 55 mg, from about 55 mg to about 60 mg, from about 60 mg to about 65 mg, from about 65 mg to about 70 mg, from about 70 mg to about 75 mg, from about 75 mg to about 80 mg, from about 80 mg to about 85 mg, from about 85 mg to about 90 mg, from about 90 mg to about 95 mg, from about 95 mg to about 100 mg, from about 100 mg to about 125 mg, from about 125 mg to about 150 mg, from about 150 mg to about 175 mg, from about 175 mg to about 200 mg, from about 200 mg to about 225 mg, from about 225 mg to about 250 mg, or from about 250 mg to about 300 mg.

A compound described herein can be present in a composition in an amount of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 120 mg, about 125 mg, about 150 mg, about 175 mg, about 200 mg, about 225 mg, about 250 mg, or about 300 mg.

A compound described herein can be administered to a subject in an amount of about 0.1 mg/kg to about 500 mg/kg, about 1 mg/kg to about 500 mg/kg, about 0.1 mg/kg to about 300 mg/kg, about 1 mg/kg to about 300 mg/kg, or about 0.1 mg/kg to about 30 mg/kg. In some embodiments, the compound disclosed herein is administered to a subject in an amount of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 120 mg/kg, about 150 mg/kg, about 160 mg/kg, about 180 mg/kg, about 200 mg/kg, about 240 mg/kg, about 250 mg/kg, about 300 mg/kg, about 350 mg/kg, about 360 mg/kg, about 400 mg/kg, about 450 mg/kg, about 500 mg/kg, or about 600 mg/kg of the subject.

A compound described herein can be administered before, during, or after the occurrence of a disease or condition, and the timing of administering the composition containing a compound can vary. For example, a compound can be used as a prophylactic and can be administered continuously to subjects with a propensity to conditions or diseases in order to lessen or reduce a likelihood of the occurrence of the disease or condition. A compound and composition can be administered to a subject during or as soon as possible after the onset of the symptoms. The administration of a compound can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein.

A compound can be administered as soon as is practical after the onset of a disease or condition is detected or suspected, and for a length of time necessary for the treatment of the disease, such as, for example, from about 1 month to about 3 months. In some embodiments, the length of time a compound can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months, about 23 months, about 2 years, about 2.5 years, about 3 years, about 3.5 years, about 4 years, about 4.5 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, about 21 years, about 22 years, about 23 years, about 24 years, or about 25 years. The length of treatment can vary for each subject.

A dosing schedule for administration of a compound described herein include, but are not limited to, once daily (QD), twice daily (BID), three times daily (TID), four times daily (QID), once weekly, twice weekly, three times weekly, once monthly, twice monthly, and once every other month.

Dosing can occur in the morning, in the afternoon or in the evening.

In some embodiments, the first compound is administered at about 150 mg/kg, about 200 mg/kg, about 250 mg/kg or about 300 mg/kg in the morning and about 100 mg/kg, about 200 mg/kg, or about 250 mg/kg in the afternoon.

Multiple therapeutic agents can be administered in any order or simultaneously. In some embodiments, a compound of the disclosure is administered in combination with, before, or after treatment with another therapeutic agent. In some embodiments, a compound of the disclosure is administered at regular intervals, such as, for example, twice daily, daily, or weekly, and the second therapeutic agent is administered intermittently or on an as-needed basis. If simultaneously, the multiple therapeutic agents can be provided in a single, unified form, or in multiple forms, for example, as multiple separate pills. The agents can be packed together or separately, in a single package or in a plurality of packages. One or all of the therapeutic agents can be given in multiple doses. If not simultaneous, the timing between the multiple doses can vary to as much as about a month.

### EXAMPLES

### Example 1: Study to Evaluate Cell Killing Activity of Rigosertib, Sotorasib, and Combination with In Vitro Assays

To test the cell killing activity of rigosertib in combination with sotorasib using in vitro assays, cell lines harboring KRASG12C and control cells harboring wild type KRAS, KRASG12D, and KRASG12V mutant RAS isotypes are tested using 96 well based dose response assays. The cell lines are originally derived from NSCLC, colorectal, and pancreatic cancers in which both the KRAS mutagenicity profile and the zygosity status (homozygous or heterozygous) are known and well characterized.

The first step is determining the dose response curve of each compound against each cell line using a 6-point concentration response assay system. Cell viability is determined after 4 days of treatment, by the following method. The cell line of interest is plated at the specified cell density into multiwell dishes (in triplicates) and the respective positive controls, negative controls, and treatments are added to the media. After incubation for a specified time, the respective reagent for the desired test method is added to the wells. The wells are then read via luminescence, fluorescence, or absorbance either immediately or after further incubation depending on the test method. These assays are done in three independent assays. The GI50 value and dose response curve are obtained to provide accurate concentrations to be used for the combination studies.

The data obtained in the 6-point dose response assay is subsequently used to determine the concentrations necessary for the combination assays. The combination assays are performed using 4 concentrations based on the GI50 curve of rigosertib and sotorasib, i.e., two concentrations lower than the GI50 value, one concentration of the GI50 value, and one concentration just above the GI50 values of each compound. The cell growth assays are run using the same 96 well protocol as used in the initial dose response assay as described above.

The sequence that the cells are treated with each compound is also very important for determining cell killing activity of rigosertib and sotorasib combination. Therefore, the combination experiments are run to determine the optimal sequence of the addition of rigosertib and sotorasib. The experimental design is as follows: 1. adding both compounds at the same time; 2. adding the first compound 24 hr prior to the second compound; 3. adding the first compound 24 hr after the second compound.

The data derived from the combination assays is used to determine the Combination Index (CI), by the Chou-Talalay equation. The derived combination index equation for two drugs is:

$$C I = \frac{{( D )}_{1}}{{( {D}_{x} )}_{1}} + \frac{{( D )}_{2}}{{( {D}_{x} )}_{2}} ,$$

where (Dₓ)₁, and (Dₓ)₂ are the concentrations of the tested substance 1 and the tested substance 2 in the single treatment that is required to decrease the cell number by x %, and (D)₁ and (D)₂ are the concentrations of the tested substance 1 in combination with the concentration of the tested substance 2 that together decreases the cell number by x %. The CI value quantitatively defines synergism (CI<1), additive effect (CI=1) and antagonism (CI>1).

As an extension to the dose response assays which determine growth inhibition, the amount of cytotoxicity based on the induction of apoptosis is also evaluated, by the activation of caspases 3/7 which is a marker for apoptosis. These studies are run under conditions based on the dose response and combination data. Combinations that show either synergistic (i.e., CI<1) or additive (i.e., CI=1) effects are then tested for the induction apoptosis. The extent of apoptosis is determined by analyzing the caspase-3 activity resulting in each combination treatment using a caspase-3 assay kit. The assay utilizes a specific substrate (N-Ac-DEVD-AFC) that is cleaved by active caspase-3, forming a highly fluorescent product. The fluorescence intensity (λ_{exc/em}=400/490 nm) is proportional to the caspase-3 activity.

### Example 2: Study to Evaluate Tumor Growth Inhibitory Activity of Rigosertib, Sotorasib, and Combination with In Vivo Assays

Once the in vitro studies are completed, the data obtained from those studies are used to validate the data using in vivo cell line derived (CDX) xenograft and patient derived xenograft (PDX) assays. The CDX assays utilize cell lines identified from the in vitro assays that resulted in either synergistic or additive responses to the combination of rigosertib and sotorasib. The specific PDX model is determined based on the KRAS mutation status of the patient samples. In addition to mutations, other perturbations of KRAS pathway can occur, to result in activation of the pathway, e.g., amplification of wild-type KRAS and secondary pathways. The secondary pathways can lead to reactivation of the KRAS pathway and thus resistance towards sotorasib treatment. To test rigosertib in combination with sotorasib, patient samples derived from cancers, such as but not limited to, NSCLC, colo-rectal, and pancreatic cancer are used. The PDX models are available from both vendors and academic collaborators. The tumors have complete genomic sequencing performed to determine the KRAS mutation status and identify any other driver mutations or amplifications in other known cell cycle and/or survival signaling pathways.

Athymic nude mice or severe combined immunodeficient (SCID) mice are implanted subcutaneously with KRAS activated cells or PDX lines. The tumors are permitted to grow to a tumor volume greater than or equal to 50 mm³. Once enough animals harbor tumors with similar tumor volumes, the mice are treated with rigosertib and sotorasib.

Rigosertib is administered at various doses and schedules to mimic the clinical use in human trials. Rigosertib is given by oral gavage at, 100 mg/kg (HED=487.8 mg), 150 mg/kg (HED=731.7 mg), 200 mg/kg (HED=975 mg), 250 mg/kg (HED=1219.8 mg), or 300 mg/kg (HED=1463.4 mg). The schedule is BID at each dose.

The schedule is further modified to mimic the scheduling in humans (to reduce possible toxicities) as follows: 150 mg/kg, 200 mg/kg, 250 mg/kg and 300 mg/kg in the morning (AM) followed by pairing with an afternoon (PM) dose (see Table 1).

TABLE 1
Rigosertib administration schedule

| AM Dose (mg/kg) | PM Dose (mg/kg) |
|---|---|
| 150, 200, 250, 300 | 100 |
| 150, 200, 250, 300 | 200 |
| 250, 300 | 200 |
| 300 | 250 |

The animals are administered sotorasib in a dose and schedule that has been previously determined in the literature to be scientifically acceptable and clinically relevant. Typically, sotorasib is given daily by oral administration at doses ranging from 10-30 mg/kg. The dose of sotorasib is chosen to achieve less than 50% tumor regression to observe synergistic or additive activity with rigosertib.

Sotorasib and rigosertib are orally administered in the morning followed by a second dose of rigosertib in the afternoon.

Assessment of the growth inhibitory activity of each compound or combination is determined by two criteria, tumor growth and increase in life span (ILS). Tumor growth is monitored by volume determination. Tumor sizes V are calculated according to the formula V=W²×L/2, where L is the length and W is the perpendicular width of the tumor and L>W. Toxicity of the combination treatments is also determined by body weights and observations of known signs of toxicity.

In addition to assessing the effects of each compound and the rigosertib and sotorasib combination on tumor growth inhibition, plasma and tumor tissue are collected to determine the pharmacokinetic (PK) and pharmacodynamic (PD) profile of each compound and the combination. Plasma levels are used in the determination of PK profiles. And the activation status of RAS downstream signaling pathways such as MAPK, ERK, and AKT is determined by immunohistochemical (IHC) and western blotting techniques. The data are used to identify plasma concentrations of each compound that results in both tumor growth inhibition and target engagement of the RAS pathway.

### Example 3: Study to Evaluate an Oral Pharmaceutical Composition Disclosed Herein for Treating a Disease in a Subject

A clinical study is performed to evaluate the safety and tolerability of a combination of sotorasib and rigosertib regimen in patients with KRAS G12C-mutated locally advanced or metastatic non-small cell lung cancer (NSCLC), as determined by an FDA-approved test, who have received/failed at least one prior systemic therapy followed by a failure on sotorasib. Approximately 15-30 patients are enrolled in the study.

### Dosage and Administration:

Study medications are administered in 28 days cycles.

Sotorasib (960 mg) is administered as an oral tablet once daily.

Rigosertib is administered orally for a total of 3 weeks, every four week cycle (three weeks on, one week off drug). Rigosertib is administered at least 2 hours after a prior meal and 1 hour before each subsequent meal according to a 3+3 dose escalation regimen described in Table 2.

TABLE 2
Rigosertib administration schedule in this study

| No. | Dose Level* | N |
|---|---|---|
| 1 | 280 mg PO qAM | 3-6 |
| | 280 mg PO qPM | |
| 2 | 560 mg PO qAM | 3-6 |
| | 280 mg PO qPM | |
| 3 | 560 mg PO qAM | 3-6 |
| | 560 mg PO qPM | |
| 4 | 840 mg PO qAM | 3-6 |
| | 560 mg PO qPM | |
| 5 | 840 mg PO qAM | 3-6 |
| | 840 mg PO qPM | |

*PO: per os; qAM: morning dose; qPM: afternoon dose

Per 3+3 dose escalation studies, the maximum tolerated dose (MTD) and recommended phase 2 dose (RP2D) are determined based on dose limiting toxicities (DLTs). After determination of the RP2D, two expansion cohorts of 10-20 are enrolled.

Expansion Cohort 1: Patients with KRAS G12C-mutated locally advanced or metastatic NSCLC, as determined by an FDA-approved test, who have received/failed at least one prior systemic therapy followed by a failure on Sotorasib.

Expansion Cohort 2: Patients with KRAS G12C-mutated locally advanced or metastatic NSCLC, as determined by an FDA-approved test, who have received/failed at least one prior systemic therapy (Sotorasib naïve).

### Study Objectives

The primary objective is to evaluate the safety and tolerability. The secondary objective is to evaluate the MTD/RP2D (≤1 out of 6 patients experiences DLT). In addition to the assessment of safety and tolerability, efficacy is measured by the overall response rate as assessed by iRECIST.

### Main Inclusion Criteria

1) Patients with KRAS G12C-mutated locally advanced or metastatic NSCLC who have failed Sotorasib as second line systemic therapy (progressed on standard of care (SoC) therapy)
2) ≥18 years old with Eastern Cooperative Oncology Group Performance Status (ECOG PS) 0-2.
3) No active CNS disease.

The Eastern Cooperative Oncology Group Scale of Performance Status (ECOG PS) is widely used to quantify the functional status of cancer patients. The PS describes the status of symptoms and functions with respect to ambulatory status and need for care. PS Grade 0 is described as fully active and able to carry on all pre-disease performance without restriction. PS Grade 1 is described as restricted in physically strenuous activity but ambulatory and able to carry out work of a light or sedentary nature. PS Grade 2 is described as ambulatory and capable of all self-care but unable to carry out any work activities (i.e., up and about more than 50% of waking hours). PS Grade 3 is described as capable of only limited self-care and is confined to a bed or chair more than 50% of waking hours. PS Grade 4 is described as completely disabled, cannot carry on any self-care, and totally confined to bed or chair. PS Grade 5 is dead.

Following determination of the RP2D a randomized, controlled phase 2 study is performed with rigosertib combination with sotorasib. Approximately 200 patients are enrolled in a 1:1 ratio to receive either sotorasib+placebo, rigosertib+placebo, or sotorasib+rigosertib.

Primary outcome is the statistical comparison between progression-free survival (PFS) rates, with overall survival (OS) as the secondary endpoint.

Efficacy Assessments: Overall response rate (ORR) is defined as the proportion of patients who achieve either a CR or a PR. Responses of complete remission (CR), partial remission (PR), mCR, SD, failure, and PD is determined by RECIST criteria. The number and percent of patients with CR, PR, mCR, SD, Failure or PD are summarized.

### Example 4: Study to Evaluate an Intravenous Pharmaceutical Composition Disclosed Herein for Treating a Disease in a Subject

A clinical study is performed to evaluate the safety and tolerability of a combination of sotorasib and rigosertib regimen in patients with KRAS G12C-mutated locally advanced or metastatic non-small cell lung cancer (NSCLC), as determined by an FDA-approved test, who have received/failed at least one prior systemic therapy followed by a failure on sotorasib. Approximately 15-30 patients are enrolled in the study.

### Dosage and Administration:

Study medications are administered in 28 days cycles.

Sotorasib (960 mg) is administered as an oral tablet once daily.

For continuous IV infusion as Rigosertib, sodium salt concentrate, 75 mg/mL which is formulated to contain 75 mg of Rigosertib sodium salt per mL (equivalent to 75 mg of Rigosertib sodium per mL) as a sterile solution in polyethylene glycol 400 (PEG 400) containing a small amount of sodium hydroxide for pH adjustment. Rigosertib.Na 75 mg/mL is packaged in single-use glass vials with Teflon-lined rubber stoppers containing 24 mL of drug product (1800 mg). Just prior to dosing, the Rigosertib concentrate (i.e. 1 glass vial, 24 mL) is diluted to a final volume of 500 ml in 0.9% sodium chloride for injection.

The initial dose of IV rigosertib given in combination is 1,200 mg/24 hours given as a continuous infusion (CIV) for days 1-3 of a 14-day cycle. The next rigosertib dose cohort is 1,500 mg/24 hours as a continuous infusion (CIV) for days 1-3 of a 14-day cycle. Dose increments in the dose escalation 3+3 study are 300 mg/24 hours each. (1,800 mg, 2,100 mg, 2,400 mg, etc.).

Per 3+3 dose escalation studies, the maximum tolerated dose (MTD) and recommended phase 2 dose (RP2D) are determined based on dose limiting toxicities (DLTs). After determination of the RP2D, two expansion cohorts of 10-20 may be enrolled.

Expansion Cohort 1: Patients with KRAS G12C-mutated locally advanced or metastatic NSCLC, as determined by an FDA-approved test, who have received/failed at least one prior systemic therapy followed by a failure on Sotorasib.

Expansion Cohort 2: Patients with KRAS G12C-mutated locally advanced or metastatic NSCLC, as determined by an FDA-approved test, who have received/failed at least one prior systemic therapy (Sotorasib naïve).

### Study Objectives

The primary objective is to evaluate the safety and tolerability. The secondary objective is to evaluate the MTD/RP2D (≤1 out of 6 patients experiences DLT). In addition to the assessment of safety and tolerability, efficacy is measured by the overall response rate as assessed by iRECIST.

### Main Inclusion Criteria

1) Patients with KRAS G12C-mutated locally advanced or metastatic NSCLC who have failed Sotorasib as second line systemic therapy (progressed on standard of care (SoC) therapy)
2) ≥18 years old with Eastern Cooperative Oncology Group Performance Status (ECOG PS) 0-2.
3) No active CNS disease

Following determination of the RP2D a randomized, controlled phase 2 study is performed with rigosertib combination with sotorasib. Approximately 200 patients are enrolled in a 1:1 ratio to receive either sotorasib+placebo, rigosertib IV+placebo, or sotorasib IV+rigosertib.

Primary outcome is the statistical comparison between progression-free survival (PFS) rates, with overall survival (OS) as the secondary endpoint.

Efficacy Assessments: Overall response rate (ORR) is defined as the proportion of patients who achieve either a CR or a PR. Responses of complete remission (CR), partial remission (PR), mCR, SD, failure, and PD is determined by RECIST criteria. The number and percent of patients with CR, PR, mCR, SD, Failure or PD are summarized.

### Embodiments

Embodiment 1. A method of treating a cancer in a subject in need thereof, the method comprising administering to the subject: a therapeutically-effective amount of a first compound, wherein the first compound is a compound of formula (I) or a pharmaceutically-acceptable salt or zwitterion thereof; and a therapeutically-effective amount of a second compound, wherein the second compound is sotorasib or a pharmaceutically-acceptable salt thereof.

Embodiment 2. The method of embodiment 1, wherein the KRAS mutation is KRASG12C, KRASG12D, or KRASG12V.

Embodiment 3. The method of embodiment 1 or 2, wherein the KRAS mutation is KRASG12C.

Embodiment 4. The method of embodiment 1 or 2, wherein the KRAS mutation is KRASG12D.

Embodiment 5. The method of embodiment 1 or 2, wherein the KRAS mutation is KRASG12V.

Embodiment 6. The method of embodiment 1, wherein the cancer is a solid cancer.

Embodiment 7. The method of embodiment 6, wherein the solid cancer is lung cancer, colorectal cancer, pancreatic cancer, or skin cancer.

Embodiment 8. The method of embodiment 1, wherein the cancer is non-small cell lung cancer.

Embodiment 9. The method of embodiment 1, wherein the non-small cell lung cancer is KRAS positive non-small cell lung cancer.

Embodiment 10. The method of embodiment 8 or 9, wherein the non-small cell lung cancer is locally advanced.

Embodiment 11. The method of any one of embodiments 8-10, wherein the non-small cell lung cancer is metastatic KRAS positive non-small cell lung cancer.

Embodiment 12. The method of any of embodiments 1-11, wherein the subject received a systemic therapy for the cancer prior to the administering.

Embodiment 13. The method of any of embodiments 1-12, wherein the subject received a systemic therapy for the cancer followed by the second compound prior to the administering.

Embodiment 14. The method of any of embodiments 1-13, wherein the cancer is a relapsing or refractory cancer.

Embodiment 15. The method of any of embodiments 1-14, wherein the first compound and the second compound are administered simultaneously.

Embodiment 16. The method of any of embodiments 1-14, wherein the first compound and the second compound are administered sequentially.

Embodiment 17. The method of embodiment 1, wherein the first compound is administered prior to the second compound.

Embodiment 18. The method of embodiment 1, wherein the second compound is administered prior to the first compound.

Embodiment 19. The method of embodiment 1, wherein the administering the first compound and the administering the second compound are at least 24 hours apart.

Embodiment 20. The method of any of embodiments 1-19, wherein the administering of the first compound is oral.

Embodiment 21. The method of embodiment 20, wherein the administering of the first compound is oral on a four-week cycle of: i) three weeks of about 280 mg per morning and about 280 mg per evening; and ii) one week of no administration.

Embodiment 22. The method of embodiment 20 or 21, wherein the administering of the first compound is oral on a four-week cycle of: i) three weeks of about 560 mg per morning and about 280 mg per evening; and ii) one week of no administration.

Embodiment 23. The method of embodiment 20 or 21, wherein the administering of the first compound is oral on a four-week cycle of: i) three weeks of about 560 mg per morning and about 560 mg per evening; and ii) one week of no administration.

Embodiment 24. The method of embodiment 20 or 21, wherein the administering of the first compound is oral on a four-week cycle of: i) three weeks of about 840 mg per morning and about 560 mg per evening; and ii) one week of no administration.

Embodiment 25. The method of embodiment 20 or 21, wherein the administering of the first compound is oral on a four-week cycle of: i) three weeks of about 840 mg per morning and about 840 mg per evening; and ii) one week of no administration.

Embodiment 26. The method of any of embodiments 1-19, wherein the administering of the first compound is intravenous.

Embodiment 27. The method of embodiment 26, wherein the administering of the first compound is a 72 hour intravenous infusion.

Embodiment 28. The method of any one of embodiments 1-27, wherein the administering of the first compound is on day 1 to 3 of a two-week cycle.

Embodiment 29. The method of any of embodiments 1-27, wherein the administering of the first compound is on day 1 to 3 of a two-week cycle for 8 cycles, then on days 1 to 3 of a four-week cycle.

Embodiment 30. The method of any of embodiments 1-27, wherein the administering of the first compound is on a four-week cycle of daily for three weeks followed by one week with no administration.

Embodiment 31. The method of embodiment 1, wherein the therapeutically-effective amount of the first compound is about 100 mg to about 1,500 mg per day.

Embodiment 32. The method of embodiment 1, wherein the therapeutically-effective amount of the first compound is about 560 mg per day.

Embodiment 33. The method of embodiment 1, wherein the therapeutically-effective amount of the first compound is about 840 mg per day.

Embodiment 34. The method of embodiment 1, wherein the therapeutically-effective amount of the first compound is about 1,120 mg per day.

Embodiment 35. The method of any of embodiments 1-34, wherein the first compound is administered on a morning and an afternoon of a day, wherein the therapeutically-effective amount of the first compound in the morning is about 150 mg/kg to about 300 mg/kg, and the therapeutically-effective amount of the first compound in the afternoon is about 100 mg/kg to about 250 mg/kg.

Embodiment 36. The method of embodiment 1, wherein the administering occurs in a morning and an evening of a day, wherein the subject is in a fasted state for administering in the morning and in a fed state for administering in the evening.

Embodiment 37. The method of embodiment 1, wherein the therapeutically-effective amount of the first compound is about 1,800 mg, and the about 1,800 mg is diluted in 0.9% saline.

Embodiment 38. The method of any preceding embodiments, wherein the administering of the second compound is oral.

Embodiment 39. The method of embodiment 1, wherein the therapeutically-effective amount of the second compound is about 960 mg per day.

Embodiment 40. The method of embodiment 1, wherein the administering of the second compound is once daily.

Embodiment 41. The method of embodiment 1, wherein the administering the first compound is oral and the administering the second compound is oral.

Embodiment 42. The method of embodiment 1, wherein the administering the first compound is intravenous and the administering the second compound is oral

Embodiment 43. The method of any one of embodiments 1-42, wherein the first compound has a formula (Ia).

Embodiment 44. The method of any one of embodiments 1-43, wherein the first compound is (E)-2-(5-((2,4,6-trimethoxystyrylsulfonyl)methyl)-2-methoxyphenylamino)acetic acid, or a pharmaceutically-acceptable salt or zwitterion thereof.

Embodiment 45. The method of any one of embodiments 1-44, wherein the first compound is sodium (E)-2-(5-((2,4,6-trimethoxystyrylsulfonyl)methyl)-2-methoxyphenylamino)acetate.

Embodiment 46. A method of treating non-small cell lung cancer in a subject in need thereof, the method comprising administering to the subject: a therapeutically-effective amount of a first compound, wherein the first compound is rigosertib or a pharmaceutically-acceptable salt or zwitterion thereof; and a therapeutically-effective amount of a second compound, wherein the second compound is sotorasib or a pharmaceutically-acceptable salt thereof.

1-46. (canceled)

47. A method of treating a cancer in a subject in need thereof, the method comprising:
administering to the subject a therapeutically-effective amount of a first compound, wherein the first compound is of formula (I) 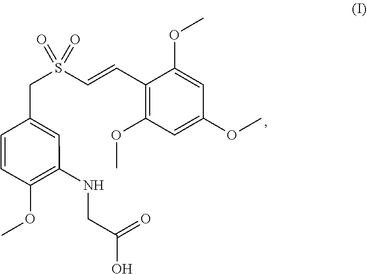 [SMILES: COc1cc(OC)c(/C=C/S(=O)(=O)Cc2ccc(OC)c(NCC(=O)O)c2)c(OC)c1]
or a pharmaceutically-acceptable salt or zwitterion thereof; and
administering to the subject a therapeutically-effective amount of a second compound, wherein the second compound is of formula (ii) 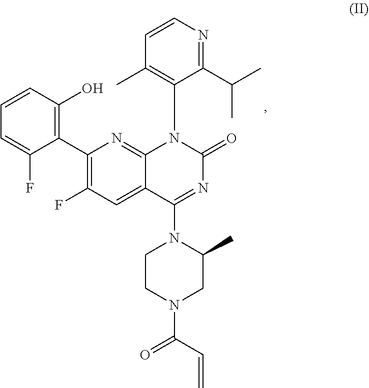 [SMILES: C=CC(=O)N1CCN(c2nc(=O)n(-c3c(C)ccnc3C(C)C)c3nc(-c4c(O)cccc4F)c(F)cc23)[C@@H](C)C1]
or a pharmaceutically-acceptable salt thereof,
wherein the cancer comprises a KRAS mutation.

48. The method of claim 47, wherein the KRAS mutation is a KRASG12C mutation.

49. The method of claim 1, wherein the KRAS mutation is a KRASG12D mutation.

50. The method of claim 47, wherein the KRAS mutation is a KRASG12V mutation.

51. The method of claim 47, wherein the cancer is a solid cancer.

52. The method of claim 47, wherein the cancer is non-small cell lung cancer.

53. The method of claim 52, wherein the non-small cell lung cancer is metastatic KRAS positive non-small cell lung cancer.

54. The method of claim 47, wherein the administering the first compound and the administering the second compound are at least 24 hours apart.

55. The method of claim 47, wherein the administering of the first compound is intravenous.

56. The method of claim 47, wherein the therapeutically-effective amount of the first compound is about 100 mg to about 1,500 mg per day.

57. The method of claim 47, wherein the therapeutically-effective amount of the first compound is about 560 mg per day.

58. The method of claim 47, wherein the therapeutically-effective amount of the first compound is about 840 mg per day.

59. The method of claim 47, wherein the therapeutically-effective amount of the first compound is about 1,120 mg per day.

60. The method of claim 47, wherein the administering the first compound occurs in a morning and an evening of a day, wherein the subject is in a fasted state for administering the first compound in the morning and in a fed state for administering the first compound in the evening.

61. The method of claim 47, wherein the therapeutically-effective amount of the second compound is about 960 mg per day.

62. The method of claim 47, wherein the administering the first compound is oral and the administering the second compound is oral.

63. The method of claim 47, wherein the administering the first compound is intravenous and the administering the second compound is oral.

64. The method of claim 47, wherein the first compound is [chemical structure]
